(19) United States
(12) Patent Application Publication
(10) Pub. No.: US 2010/0004745 A1
(43) Pub. Date: Jan. 7, 2010

(54) Fusing Bone

(75) Inventors: Richard J. Deslauriers, Woodbury, CT (US); Christopher P. Ames, Mill Valley, CA (US); Wade M. Ceola, Springfield, MO (US); Michael W. Hasz, Reston, VA (US)

Correspondence Address: FISH & RICHARDSON P.C., P.O. BOX 1022, MINNEAPOLIS, MN, 55440-1022, US

(73) Assignee: DOCTORS RESEARCH GROUP, INC.

(21) Appl. No.: 12/166,304

(22) Filed: Jul. 1, 2008

(51) Int. Cl. A61F 2/44

(52) U.S. Cl. 623/17.12; 623/17.11; 623/17.16

(57) ABSTRACT

Systems and techniques for fusing bone or bone fragments. In one aspect, an apparatus includes an interbody member holder comprising a connector and a channel arranged to form a flow connection between the interbody member holder and an interior channel of an interbody member held on the connector.

### BACKGROUND

This disclosure relates to the fusing of bone or bone fragments, such as the fusing of vertebrae in the spine.

There are many circumstances in which bones or bone fragments are fused, including fractures, joint degeneration, abnormal bone growth, infection, and the like. For example, indications for spinal fusion include degenerative disc disease, spinal disc herniation, discogenic pain, spinal tumors, vertebral fractures, scoliosis, kyphosis, spondylolisthesis, spondylosis, Posterior Rami Syndrome, other degenerative spinal conditions, and other conditions that result in instability of the spine.

### SUMMARY

Systems and techniques for fusing bone or bone fragments are described. In one aspect, an apparatus includes an interbody member holder comprising a connector and a channel arranged to form a flow connection between the interbody member holder and an interior channel of an interbody member held on the connector.

This and other aspects can include one or more of the following features. The channel can be defined within the connector. The connector can be a male connector dimensioned to be insertable into a female receptacle of the interbody member. The connector can include a threaded surface and/or at least a portion of a fitting.

The interbody member holder can include a release mechanism for releasing the fitting. The connector can include a surface oriented to contact and oppose a complementary surface of the interbody member held on the connector to allow rotation of the interbody member. The apparatus can include an extruder.

In another aspect, a device includes an interbody member defining an interior network of flow channels and comprising a connection site for forming a junction with an interbody member holder. A first of the flow channels can open at a side of the interbody member and a second of the flow channels can open at the junction.

This and other aspects can include one or more of the following features. The connection site can include a receptacle dimensioned to receive a connection element of the interbody member. The receptacle can be connected to the interior network of flow channels. The interbody member can be dimensioned to be positioned in a predetermined spatial relationship relative to a bone or bone fragment at a surgical site. One or more flow channels of the interior network can be configured to preferentially direct a flow received from the second of the flow channels out a side of the interbody member that is opposed to or in contact with the bone or the bone fragment with the interbody member positioned in the predetermined relationship relative thereto. The connection site can include a threaded surface. The connection site can include a portion of a compression fitting.

In another aspect, a system includes an interbody member defining an interior flow network of one or more channels, and an interbody member holder. The interbody member holder can include a connector configured and dimensioned to hold the interbody member and a channel connectable to the flow network of the interbody member.

This and other aspects can include one or more of the following features. connector of the interbody member holder can define the channel. The system can include an extruder movable within the interbody member holder.

In another aspect, a method includes inserting an interbody member into a space between bones or bone fragments, and extruding a fixative through the interbody member to contact the bones or the bone fragments.

This and other aspects can include one or more of the following features. Inserting the interbody member can include spacing spinal vertebrae using a cage. Extruding the fixative can include contacting the fixative to spinal vertebrae. Extruding the fixative can include substantially filling the intervertebral space between the spinal vertebrae with the fixative so that the fixative, upon hardening, forms a disc-shaped solid member.

In another aspect, a method includes fusing vertebrae of the spine without hardware by flowing a liquid polymeric fixative into an intervertebral space. The polymeric fixative adheres to opposing surfaces of the spinal vertebrae and bears at least some of the physiological loads therebetween when hardened.

This and other aspects can include one or more of the following features. Flowing the liquid polymeric fixative can include extruding the liquid polymeric fixative through a cage. Fusing the vertebrae can include approaching the vertebrae using a transverse approach. Fusing the vertebrae can include approaching the vertebrae using a transverse approach. Flowing the liquid polymeric fixative can include preferentially directing the flow of the liquid polymeric fixative toward faces of the vertebrae.

In another aspect, a spinal fusion includes a solid, load bearing polymeric fixative fixed to opposing faces of spinal vertebrae without hardware.

This and other aspects can include one or more of the following features. The solid, load bearing polymeric fixative can include a polyurethane. A cage can be disposed between the opposing faces of the spinal vertebrae. The solid, load bearing polymeric fixative can include a disc-shaped solid member that is dimensioned by the opposing faces of the spinal vertebrae.

The details of one or more implementations are set forth in the accompanying drawings and the description below. Other features and advantages will be apparent from the description and drawings and from the claims.

### DESCRIPTION OF DRAWINGS

FIG. 1 is a representation of a site that is a candidate for bone fusion.

FIG. 2 is an enlarged schematic representation of an intervertebral space at the site of FIG. 1.

FIG. 3 is a schematic representation of the intervertebral space after removal of the entirety of an intervertebral disc.

FIG. 4 is a schematic representation of a system for fusing bone or bone fragments.

FIGS. 5, 7 are schematic representations of interbody members.

FIGS. 6, 8 are sectional views of the interbody members represented in FIGS. 5, 7 taken along section AA of FIG. 4.

FIG. 9 is a schematic representation of an intervertebral space during insertion of an interbody member.

FIG. 10 is a schematic representation of an intervertebral space after rotation of an inserted interbody member.

FIG. 11 is a schematic representation of the use of a fixative extruder to extrude fixative from an interbody member holder.

FIGS. 12-13 are schematic representations of the extrusion of a fixative from an interbody member holder, through an interbody member, and into an intervertebral space.

FIG. 14 is a schematic representation of an intervertebral space after withdrawal of an interbody member holder.

FIG. 15 is a representation of the site of FIG. 1 after bone fusion.

Like reference symbols in the various drawings indicate like elements.

### DETAILED DESCRIPTION

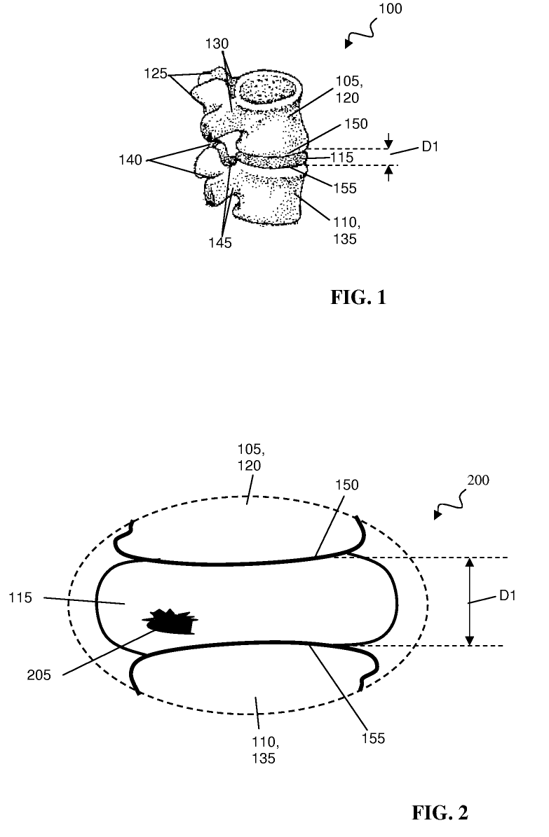

FIG. 1 is a representation of a site 100 that is a candidate for bone fusion. Site 100 includes a first vertebra 105, a second vertebra 110, and an intervertebral disc 115. Vertebra 105 includes a body 120 that is joined to transverse processes 125 by pedicles 130. Vertebra 105 includes a body 135 that is joined to transverse processes 140 by pedicles 145. Body 120 has a lower face 150. Body 135 has an upper face 155. Before fusion, faces 150, 155 oppose one another and are separated by a distance D1. The volume between faces 150, 155 is occupied by intervertebral disc 115.

FIG. 2 is an enlarged schematic representation of the volume between faces 150, 155 at site 100, namely, intervertebral space 200. Intervertebral space 200 is occupied by intervertebral disc 115. Intervertebral disc 115 can include one or more indications for spinal fusion such as a defect 205.

Site 100 can be accessed and prepared for fusion of vertebrae 105, 110 in a variety of different ways. For example, anterior, posterior, and transverse approaches in open and closed (e.g., endoscopic) surgical procedures can be used. All or a portion of disc 115 can be removed from intervertebral space 200 using diskectomy techniques, including mechanical, thermal, electrical, and/or chemical techniques. Examples of these techniques include grinding, scraping, ablation, heating, cooling, etching, digestion, and the like.

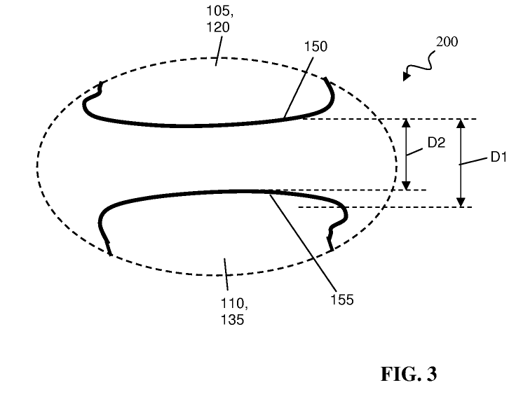

FIG. 3 is a schematic representation of intervertebral space 200 after removal of the entirety of intervertebral disc 115. As shown, intervertebral space 200 has been emptied. In many cases, after removal of intervertebral disc 115, faces 150, 155 will generally move closer together to be separated by a distance D2 that is shorter than distance D1.

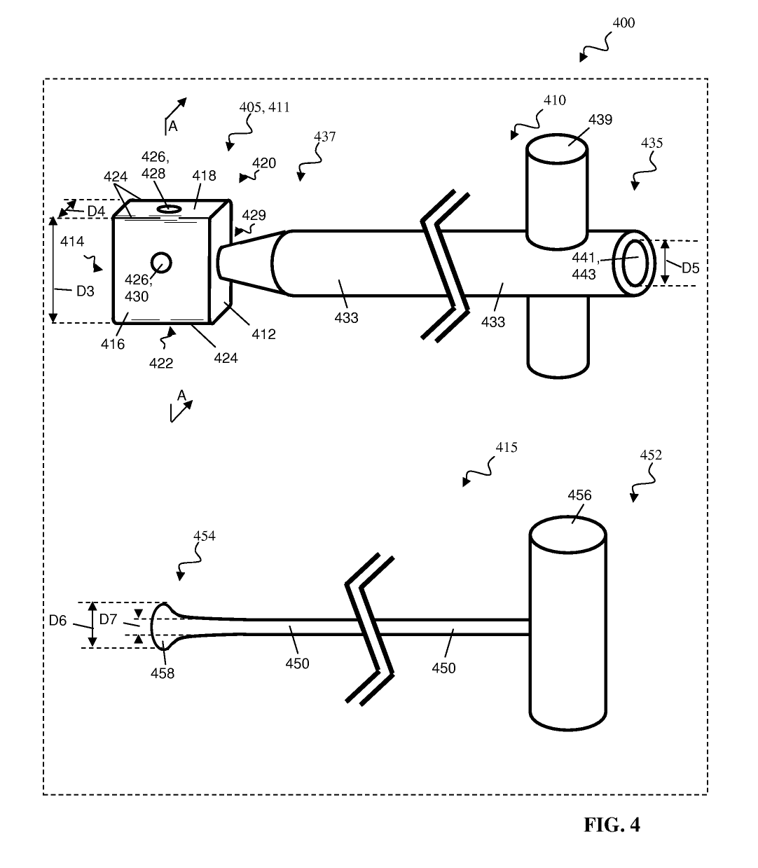

FIG. 4 is a schematic representation of a system 400 for fusing bone or bone fragments, such as vertebrae 105, 110. System 400 includes an interbody member 405, an interbody member holder 410, and a fixative extruder 415.

Interbody member 405 is a element that is shaped and dimensioned to be positioned in a physiological space defined between bones, or bone fragments, that are to be fused. For example, interbody member 405 can be an intervertebral cage 411. Cage 411 is a generally rectangular member that includes a proximal face 412, a distal face 414, and a collection of side faces 416, 418, 420, 422. Pairs of side faces 416, 418, 420, 422 meet one another at a collection of rounded edges 424.

Cage 411 defines a network 426 of one or more flow channels 428, 430. Flow channel 428 opens at side face 418. Flow channel 430 opens at side face 416. Additional flow channels can open at side faces 416, 418, 420, 422, as well as at proximal face 412 and distal face 414. As discussed further below, network 426 can be removably connected to a channel within interbody member holder 410 for the flow of liquids, gasses, semi-liquids, suspensions, mixtures, and the like therebetween.

Cage 411 is contacted and held by interbody member holder 410 at a junction 429 at proximal face 412. Junction 429 can include the flow connection between network 426 and a channel within interbody member holder 410.

Cage 411 can be dimensioned for implantation in an intervertebral space 200 of a patient. For example, side faces 418, 422 can be separated by a distance D3 that is larger than the separation distance D2 between faces 150, 155 of vertebrae 105, 110 after removal of intervertebral disc 115. Side faces 416, 420 can be separated by a distance D4 that is smaller that both distance D3 and distance D2 to facilitate insertion of cage 411 between faces 150, 155 after removal of intervertebral disc 115, as discussed further below.

In some implementations, cage 411 can have sufficient mechanical strength to bear the physiological scale loads associated with a long term intervertebral implantation without assistance. In other implementations, cage 411 can have sufficient mechanical strength to separate faces 150, 155 of a pair of vertebrae 105, 110 for relatively short times (e.g., during a procedure that fuses vertebrae 105, 110) but lack the mechanical strength necessary to bear the physiological scale loads associated with movement and longer term implantation.

Cage 411 can include metallic, polymeric, and/or a ceramic materials. For example, cage 411 can include stainless steel, alumina, titanium, or the like. In some implementations, cage 411 can include a polymeric material, such as a polyurethane. For example, cage 411 can be the polymerization product of a polyisocyanate and a polyol and/or a polyamine. Example polyisocyanates include diisocyanates, aliphatic, alicyclic, cycloaliphatic, and/or aromatic polyisocyanates. Example polyols include synthetic polyols, naturally occurring polyols, and/or hydroxylated synthetic and/or naturally occurring species.

In some implementations, cage 411 can include a composite of a polymer and one or more other components, such as a ceramic component. Example ceramic components include hydroxyapatite, demineralized bone, mineralized bone, calcium carbonate, calcium sulfate, sodium phosphate, calcium aluminate, calcium phosphate, calcium carbonate, calcium phosphosilicate, silica, baria-boralumino-silicate glass, and the like.

In some implementations, cage 411 can be the product of polymerizing a mixture of 3.89 g of castor oil polyol (e.g., Caspol 1962 available from CasChem, Inc.), 0.145 g of ricinoleic acid, 4.34 g of aromatic isocyanate (e.g., Mondur MRS-2 available from Bayer AG), 0.87 g of castor oil polyol(diol) (e.g., Caspol 1842 available from CasChem, Inc.), 0.58 g of castor oil polyol(diol) (e.g., Caspol 5001 available from CasChem, Inc.), and 0.17 g propylene carbonate in the presence of 0.027 g of potassium octoate-catalyst (e.g., Dabco T-45 available from Air Products) and 0.0026 g of tin catalyst (e.g., Cotin 1707 available from CasChem, Inc.). In some implementations, 4.27 g of calcium carbonate can be added to such a mixture. In other implementations, between 1.30 and 1.40 g of calcium carbonate and 2.8 to 3.0 g of barium sulfate can be added to such a mixture. In some implementations, cage 411 includes KRYPTONITE BONE CEMENT™, available from DOCTORS RESEARCH GROUP, INC.™ (Plymouth, Conn.).

Cage 411 can be a solid and/or a porous member. For example, cage 411 can include open and/or closed surface pores dimensioned to promote adhesion and/or ingrowth of bone or other tissues into cage 411. Porosity in cage 411 can be induced and/or tailored, e.g., using the ceramic components discussed above.

Interbody member holder 410 is configured to hold interbody member 405 for manipulation into and around a physiological space by a user. For example, interbody member holder 410 can hold an interbody member 405 during a closed surgical procedure such as a percutaneous spinal fusion. Interbody member holder 410 includes a generally elongate shaft 433 that extends longitudinally between a proximal end 435 and a distal end 437. Shaft 433 defines a channel 441 that is connectable to network 426 of interbody member 405 for flow therebetween. In the illustrated implementation, channel 441 opens at an opening 443 at proximal end 435 and has a diameter D5. Proximal end 435 of shaft 433 is fixed to a handle 439. Handle 439 allows a user to manipulate interbody member holder 410, as well as any interbody member 405 held thereby.

Distal end 437 of shaft 433 contacts and holds cage 411 at junction 429. Junction 429 can include the flow connection between network 426 in cage 411 and channel 441 within interbody member holder 410, as discussed further below.

Fixative extruder 415 is adapted to extrude fixative from channel 441 within interbody member holder 410, into network 426 of cage 411, and out one or more openings in one or more of faces 412, 414, 416, 418, 420, 422. Fixative extruder 415 includes a generally elongate shaft 450 that extends longitudinally between a proximal end 452 and a distal end 454. Proximal end 452 of shaft 450 is fixed to a handle 456. Handle 439 allows a user to manipulate fixative extruder 415 and apply pressure to fixative in channel 441. Distal end 454 of shaft 450 includes and terminates in a crown 458. Crown 458 has a diameter D6 that is larger than a diameter D7 of shaft 450 in the vicinity of crown 458. Further, diameter D6 is dimensioned to be snugly received within channel 441 of interbody member holder 410 to extrude fixative from channel 441 within interbody member holder 410 into and through network 426 of cage 411, as discussed further below.

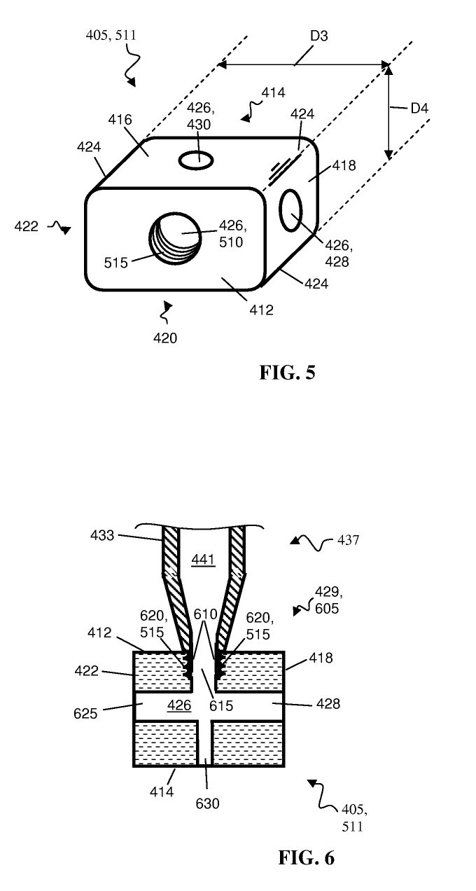

FIG. 5 is a schematic representation of an implementation of an interbody member 405, namely, a cage 511. A channel 510 opens at proximal face 412 of cage 511. In the vicinity of this opening, channel 510 includes threads 515 for forming junction 429 between cage 511 and interbody member holder 410. Channel 510 is connected to flow channel network 426 as part of the flow connection between network 426 and channel 441 when cage 511 is joined to interbody member holder 410.

FIG. 6 is sectional view taken along section AA of FIG. 4 and shows cage 511 joined to an implementation of interbody member holder 410 at junction 429. As shown, the illustrated interbody member holder 410 includes a connector 605 that extends distally from a terminal distal end of shaft 433. Connector 605 is generally tubular and includes a wall 610 that defines a channel 615. The outer surface of wall 610 includes threads 620 that are dimensioned to mate with threads 515 within channel 510. This mating joins cage 511 to interbody member holder 410 and connects channel 441 within interbody member holder 410 to flow channel network 426 within cage 511. The number and positioning of threads 620, 515 can be selected to allow rotation of cage 511 by interbody member holder 410 in one direction when positioned in an intervebral space but yet allow interbody member holder 410 to be detached from cage 511 by rotation in the other direction.

With cage 511 thus joined to interbody member holder 410, fixative can be extruded from channel 441 within interbody member holder 410 through channel 615. From channel 615, the fixative can further be extruded into network 426. From within network 426, the fixative can further be extruded through flow channel 428 and out side face 418, through a flow channel 625 out side face 422, and through a flow channel 630 and out side face 414. In some implementations, additional channels can direct fixative to be extruded out one or more of faces 412, 416, 420 as well.

The dimensioning and arrangement of channels that form network 426 can be used to direct extrusion. For example, in the illustrated implementation, channels 428, 625 are larger than channel 630 to preferentially direct flow out side faces 418, 422. Such a flow can help ensure that fixative contacts faces 150, 155 of vertebrae 105, 110 during the fusing of bone.

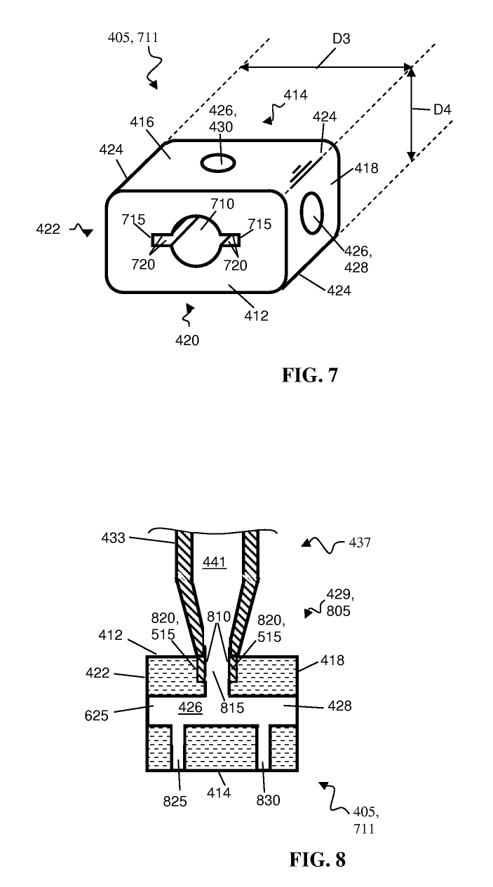

FIG. 7 is a schematic representation of an implementation of an interbody member 405, namely, a cage 711. A channel 710 opens at proximal face 412 of cage 711. In the vicinity of this opening, channel 710 includes a pair of slit-like receptacles 715 for forming junction 429 (FIG. 4) between cage 711 and interbody member holder 410. Receptacles 715 each include a pair of opposing faces 720. Faces 720 are arranged to contact and oppose faces of complementary wing members of a connector of an interbody member holder to facilitate rotation of cage 711 when cage 711 is positioned in an intervebral space. Channel 710 is connected to flow channel network 426 and forms the flow connection between network 426 and channel 441 of interbody member holder 410 when cage 711 is joined to interbody member holder 410.

FIG. 8 is sectional view taken along section AA of FIG. 4 and shows cage 711 joined to an implementation of interbody member holder 410 at junction 429. As shown, the illustrated interbody member holder 410 includes a connector 805 that extends distally from a terminal distal end of shaft 433. Connector 805 is generally tubular and includes a wall 810 that defines a channel 815.

A pair of wings 820 extend radially outward from wall 810 and are dimensioned to be received in receptacles 715 of channel 710. With wings 820 received in receptacles 715, channel 441 within interbody member holder 410 is connected to flow channel network 426 within cage 711. In some implementations, wings 820 can be dimensioned to form a compression or other fitting with receptacles 715. Such a fitting can allow manipulation—including rotation—of cage 711 by interbody member holder 410 when cage 711 is positioned in an intervebral space. In other implementations, additional members can be used to ensure that junction 429 has sufficient mechanical integrity to allow manipulation in such circumstances.

With cage 711 joined to interbody member holder 410, fixative can be extruded from channel 441 within interbody member holder 410 through channel 815. From channel 815, the fixative can further be extruded into network 426. From within network 426, the fixative can further be extruded through flow channel 428 and out side face 418, through flow channel 625 and out side face 422, and through a pair of flow channels 825, 830 out side face 414. In some implementations, additional channels can direct fixative to be extruded out one or more of faces 412, 416, 420 as well.

The dimensioning and arrangement of channels that form network 426 can be used to direct extrusion. For example, in the illustrated implementation, the sectional area of each of channels 428, 625 is larger than the total sectional area of channels 825, 830. Further, channels 825, 830 are displaced so as not to be aligned with channel 815 of interbody member holder 410. These measures can preferentially direct flow out side faces 418, 422. Such a flow can help ensure that fixative contacts faces 150, 155 of vertebrae 105, 110 during the fusing of bone.

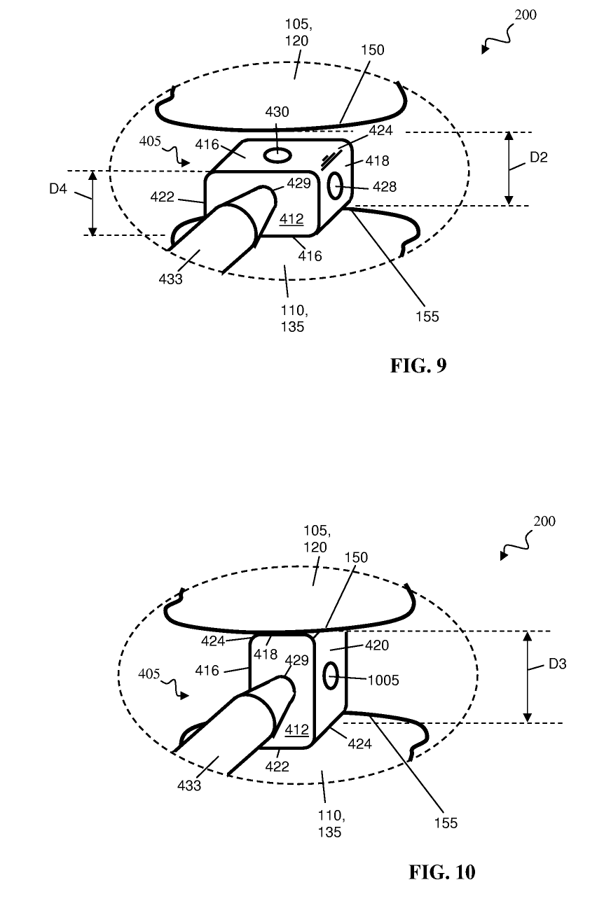

FIG. 9 is a schematic representation of intervertebral space 200 during insertion of interbody member 405. For example, interbody member 405 can be one of cages 411, 511, 711. Interbody member 405 can be inserted via an anterior, posterior, or transverse approach to intervertebral space 200. As shown, interbody member 405 can be inserted with side face 416 opposing face 150 of vertebra 120 and with side face 420 opposing face 155 of vertebra 110. Such an insertion is facilitated by distance D4 being smaller than distance D2.

After such an insertion, interbody member 405 can be rotated. For example, when interbody member 405 is mounted on interbody member holder 410 (FIG. 4), a physician or other user can use handle 439 to rotate both holder 410 and member 405, e.g., by 90°. Other interbody member holders can rotate member 405 in different ways. For example, an interbody member can include a motor, a coiled spring, a ratcheting mechanism, or other element for rotating interbody member 405.

FIG. 10 is a schematic representation of intervertebral space 200 after rotation of inserted interbody member 405. As shown, rotation of interbody member 405 moves side face 418 into opposition and contact with face 150 of vertebra 120 and side face 422 into opposition and contact with face 155 of vertebra 110. Since distance D3 is generally larger than distance D2, this rotation generally increases the separation between faces 150, 155. Indeed, in some implementations, distance D3 can approximate the separation distance D1 between faces 150, 155 prior to removal of intervertebral disc 115.

In some instances, faces 150, 155 can be separated by a retractor or other device (not shown) prior to rotation of interbody member 405. Rounded edges 424 can facilitate rotation of interbody member 405 by reducing the need for additional separation of faces 150, 155 during rotation.

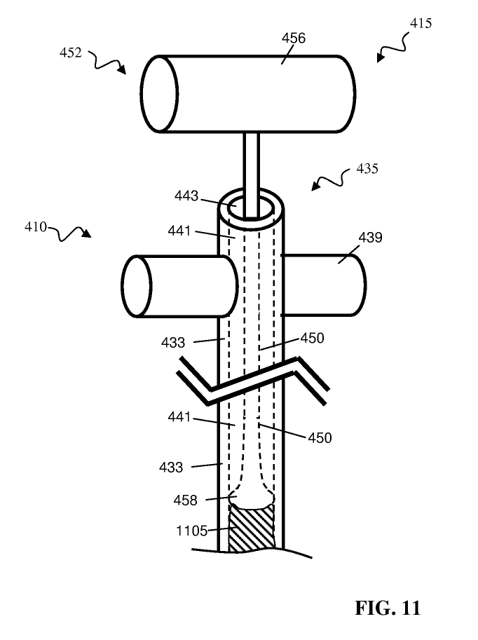

FIG. 11 is a schematic representation of the use of fixative extruder 415 to extrude fixative from interbody member holder 410. A fixative 1105 can be loaded into channel 441 of interbody member holder 410 in a variety of ways. For example, fixative 1105 can be poured, driven under pressure, or otherwise inserted into opening 443 of channel 441 prior to insertion of interbody member 405 into intervertebral space 200. Crown 458 of fixative extruder 415 can be inserted into channel 441 and translated distally using handle 456. As discussed above, crown 458 is dimensioned to be snugly received within channel 441 of interbody member holder 410 and distal translation of crown 458 will drive fixative 1105, along with any air in channel 441, distally. Air in channel 441 can be cleared, e.g., by elevating distal end 437 during loading. In many instances, fixative 1105 has a relatively high viscosity. The dimensioning of fixative extruder shaft 450 to have a diameter D7 that is smaller than diameter D5 of channel 441 can facilitate distal translation of crown 458 (FIG. 4). In particular, contact friction between fixative extruder 415 and the wall of channel 441 is reduced.

Other examples of loading fixative 1105 include accessing channel 441 from other directions and/or using containers to load fixative 1105 in channel 441. For example, one or more containers (such as a bag, a cartridge, an ampule, or the like) can contain one or more components of fixative 1105. System 400 can include a mechanism for accessing a contained component and mixing fixative 1105 within channel 441. For example, the terminal end of crown 458 can include blades, tines, or other members that can be used to open a container and mix components of fixative 1105 in channel 441, e.g., by rotating fixative extruder 415.

Once loaded, channel 441 can act as a source or reservoir of fixative 1105 for delivery of fixative 1105 into intervertebral space 200.

In some implementations, fixative 1105 includes a polymer such as a polyurethane. For example, fixative 1105 can be the polymerization product of a polyisocyanate and a polyol and/or a polyamine. Example polyisocyanates include diisocyanates, aliphatic, alicyclic, cycloaliphatic, and/or aromatic polyisocyanates. Example polyols include synthetic polyols, naturally occurring polyols, and/or hydroxylated synthetic and/or naturally occurring species.

In some implementations, fixative 1105 can include a composite of a polymer and another component, such as a ceramic component. Example ceramic components include hydroxyapatite, demineralized bone, mineralized bone, calcium carbonate, calcium sulfate, sodium phosphate, calcium aluminate, calcium phosphate, calcium carbonate, calcium phosphosilicate, silica, baria-boralumino-silicate glass, and the like.

In some implementations, fixative 1105 can be a mixture of 3.89 g of castor oil polyol (e.g., Caspol 1962 available from CasChem, Inc.), 0.145 g of ricinoleic acid, 4.34 g of aromatic isocyanate (e.g., Mondur MRS-2 available from Bayer AG), 0.87 g of castor oil polyol(diol) (e.g., Caspol 1842 available from CasChem, Inc.), 0.58 g of castor oil polyol(diol) (e.g., Caspol 5001 available from CasChem, Inc.), and 0.17 g propylene carbonate in the presence of 0.027 g of potassium octoate-catalyst (e.g., Dabco T-45 available from Air Products) and 0.0026 g of tin catalyst (e.g., Cotin 1707 available from CasChem, Inc.). In some implementations, 4.27 g of calcium carbonate can be added to such a mixture. In other implementations, between 1.30 and 1.40 g of calcium carbonate and 2.8 to 3.0 g of barium sulfate can be added to such a mixture. In some implementations, fixative 1105 includes KRYPTONITE BONE CEMENT™, available from DOCTORS RESEARCH GROUP, INC.™ (Plymouth, Conn.).

By selecting the appropriate composition, the properties of fixative 1105 can be tailored to the systems and techniques described herein. For example, the composition can be tailored to begin polymerization upon contact of the selected polyisocyanate and polyol and/or polyamine components. After contact, the components can transition from a flowable, liquid state, through a viscous, taffy-like state, to a hardened solid state. The time for this transition can be tailored to the operational circumstances. For example, the components can be flowable under pressures achievable using fixative extruder 415 for a sufficient time to allow a surgeon or other user to perform the fusion techniques described herein.

As another example, the composition can be tailored to adhere to bone with sufficient integrity to bear the physiological-scale rotational, shear, compressive, and any other loads at the site of fusion.

As yet another example, the composition can be tailored to achieve a desirable porosity, promote bone ingrowth, and/or achieve a selected rate of degradation at the site of fusion. For example, the composition can be tailored to bond to adjacent bone without the formation of a inflammatory field or subsequent formation of a fibrous capsule such that direct osteoblast and ostoeclast infiltration can occur. Additionally ceramic components can act to maintain pH in the vicinity of the composition, while being compatible with or even inducing bone growth.

Further detail regarding such compositions, and the tailoring of the mechanical properties of such compositions, can be found in U.S. patent application Ser. No. 10/808,188, which has been published as U.S. Patent Publication No. 2005/0031578, and U.S. patent application Ser. No. 10/771,736, which has been published as U.S. Patent Publication No. 2005/0027033, the contents of both of which are incorporated herein by reference.

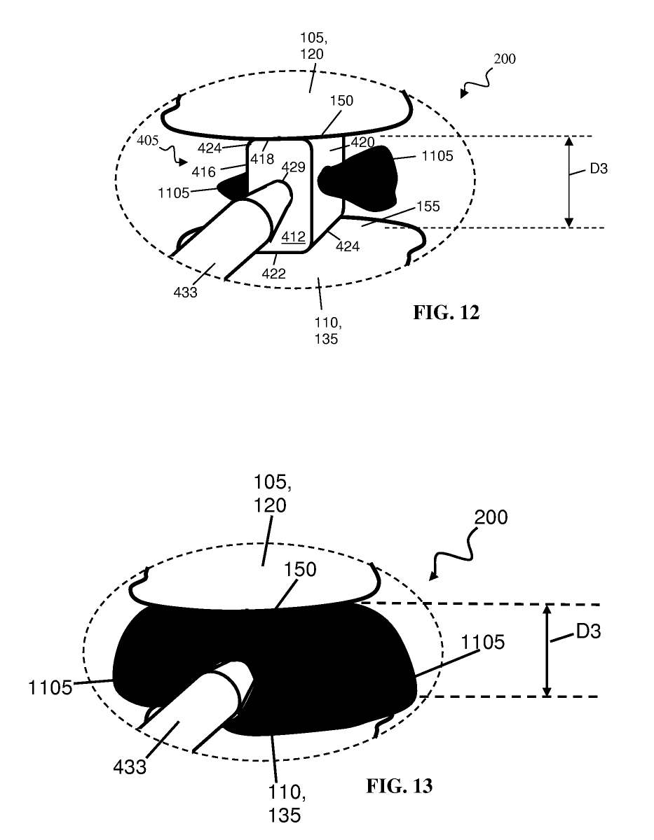

FIGS. 12-13 are schematic representations of the extrusion of fixative 1105 from interbody member holder 410, through interbody member 405, and into intervertebral space 200. At some point, distal translation of crown 458 extrudes fixative 1105 from channel 441 and across junction 429. The extruded fixative enters network 426 and is directed, via flow channels therein, out of one or more of faces 412, 414, 416, 418, 420, 422 of interbody member 405. In the illustrated implementation, fixative 1105 is extruded out side face 418 into contact with face 150 of vertebra 105, out side face 422 into contact with face 155 of vertebra 110, and out side faces 416, 420 into intervertebral space 200. As extrusion progresses, more and more fixative 1105 enters intervertebral space 200.

Further, given the flowability of fixative 1105, fixative 1105 can flow to roughly conform to the size and shape of intervertebral space 200. In effect, fixative 1105 can form a replacement intervertebral disc that has been dimensioned by the very same physiology that is being treated.

After a sufficient volume of fixative 1105 enters intervertebral space 200, interbody member holder 410 can be detached from interbody member 405 and withdrawn from the body. For example, when interbody member 405 is cage 511 that includes a threaded channel 510, interbody member holder 410 can be detached from interbody member 405 by rotation. As another example, when interbody member 405 is cage 711 that includes a compression or other fitting, interbody member holder 410 can be detached from interbody member 405 by releasing the fitting. For example, in some instances, pressure on interbody member 405 from faces 150, 155 of vertebrae 105, 110 can allow a compression fitting to be released by pulling interbody member holder 410 away from interbody member 405. In other instances, interbody member holder 410 can include a release mechanism for releasing such a fitting.

In some implementations, an outer surface of the distal end 437 of interbody member holder 410 can be coated with an adhesion-resistant film to facilitate withdrawal of interbody member holder 410 from the body. For example, distal end 437 of interbody member holder 410 can be coated with a liquid or particulate film that reduces or eliminates adhesion between fixative 1105 and distal end 437. Such a particulate film can include polymeric and/or ceramic particles. Example polymeric particles include polyurethane particles and/or particles of polyurethane composites. Example ceramic particles include hydroxyapatite particles, demineralized bone particles, mineralized bone particles, calcium carbonate particles, calcium sulfate particles, sodium phosphate particles, calcium aluminate particles, calcium phosphate particles, calcium phosphosilicate particles, silica particles, baria-boralumino-silicate glass particles, and the like.

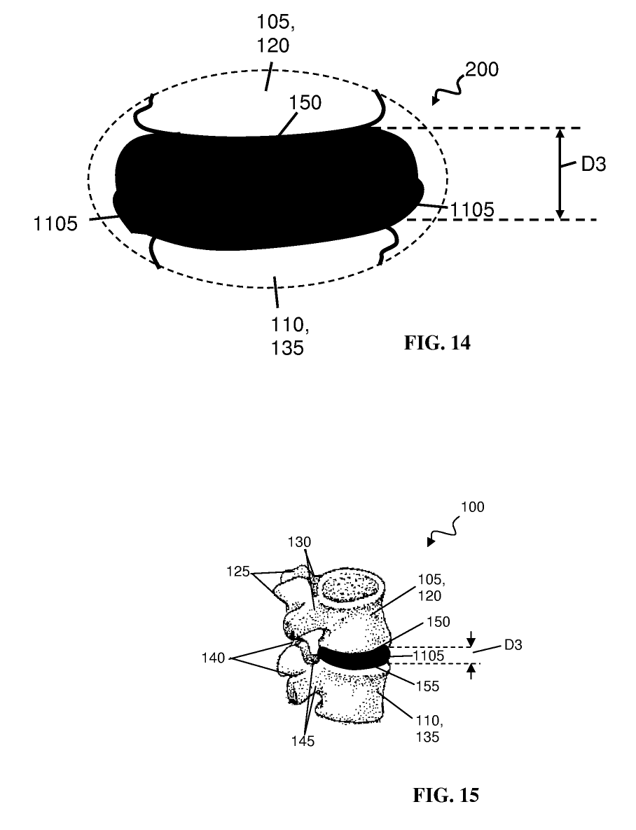

FIG. 14 is a schematic representation of intervertebral space 200 after withdrawal of interbody member holder 410. As shown, a sufficient amount of fixative 1105 can be extruded into intervertebral space 200 to mimic an intervertebral disc. Further, faces 150, 155 of vertebrae 105, 110 are a distance D3 apart and, as fixative 1105 hardens, supported by fixative 1105 as well as interbody member 405. Further, as fixative 1105 hardens, it adheres (or “fixes”) to faces 150, 155 of vertebrae 105, 110. In other words, fixative 1105 hardens to form a load-bearing member that fuses vertebrae 105, 110 and prevents relative motion, including rotation, therebetween.

FIG. 15 is a representation of site 100 after such a bone fusion. As shown, vertebrae 105, 110 have been fused. Fixative 1105 is in the intervertebral space 200 between faces 150, 155 and faces 150, 155 are held a distance D3 apart. Distance D3 can approximate the distance D1 between faces 150, 155 prior to the fusion. Further, vertebrae 105, 110 have been fused without hardware, such as pedicle screws, rods, or the like. Indeed, transverse processes 125 need not be accessed at all during the fusion.

A number of implementations have been described. Nevertheless, it will be understood that various modifications may be made. For example, although the illustrated junctions between an interbody member and an interbody member holder are formed using a male connector on the interbody member holder and a female receptacle on the interbody member, junctions can also be formed using a female receptacle on the interbody member holder and a male connector on the interbody member.

As another example, although system 400 is schematically represented using relatively simple mechanical members, a variety of changes can be made. For example, pressure for extruding a fixative can be applied using, e.g., compressed gas, a hydraulic system, loaded springs, or the like. Other interbody members can be used. Bones and bone fragments at other sites can be fused.

Accordingly, other implementations are within the scope of the following claims.

1. An apparatus comprising:
an interbody member holder comprising a connector and a channel arranged to form a flow connection between the interbody member holder and an interior channel of an interbody member held on the connector.

2. The apparatus of claim 1, wherein the channel is defined within the connector.

3. The apparatus of claim 1, wherein the connector comprises a male connector dimensioned to be insertable into a female receptacle of the interbody member.

4. The apparatus of claim 1, wherein the connector comprises a threaded surface.

5. The apparatus of claim 1, wherein the connector comprises at least a portion of a fitting.

6. The apparatus of claim 5, wherein the interbody member holder further comprises a release mechanism for releasing the fitting.

7. The apparatus of claim 1, wherein the connector comprises a surface oriented to contact and oppose a complementary surface of the interbody member held on the connector to allow rotation of the interbody member.

8. The apparatus of claim 1, further comprising an extruder.

9. A device comprising:
an interbody member defining an interior network of flow channels and comprising a connection site for forming a junction with an interbody member holder,
wherein a first of the flow channels opens at a side of the interbody member and a second of the flow channels opens at the junction.

10. The device of claim 9, wherein the connection site comprises a receptacle dimensioned to receive a connection element of the interbody member.

11. The device of claim 10, wherein the receptacle is connected to the interior network of flow channels.

12. The device of claim 9, wherein:
the interbody member is dimensioned to be positioned in a predetermined spatial relationship relative to a bone or bone fragment at a surgical site; and
one or more flow channels of the interior network are configured to preferentially direct a flow received from the second of the flow channels out a side of the interbody member that is opposed to or in contact with the bone or the bone fragment with the interbody member positioned in the predetermined relationship relative thereto.

13. The device of claim 9, wherein the connection site comprises a threaded surface.

14. The device of claim 9, wherein the connection site comprises a portion of a compression fitting.

15. A system comprising:
an interbody member defining an interior flow network of one or more channels; and
an interbody member holder comprising
a connector configured and dimensioned to hold the interbody member, and
a channel connectable to the flow network of the interbody member.

16. The system of claim 15, wherein the connector of the interbody member holder defines the channel.

17. The system of claim 15, further comprising an extruder movable within the interbody member holder.

18. A method comprising:
inserting an interbody member into a space between bones or bone fragments; and
extruding a fixative through the interbody member to contact the bones or the bone fragments.

19. The method of claim 18, wherein:
inserting the interbody member comprises spacing spinal vertebrae using a cage; and
extruding the fixative comprises contacting the fixative to spinal vertebrae.

20. The method of claim 19, wherein extruding the fixative comprises substantially filling the intervertebral space between the spinal vertebrae with the fixative so that the fixative, upon hardening, forms a disc-shaped solid member.

21. A method comprising:
fusing vertebrae of the spine without hardware by flowing a liquid polymeric fixative into an intervertebral space, wherein the polymeric fixative adheres to opposing surfaces of the spinal vertebrae and bears at least some of the physiological loads therebetween when hardened.

22. The method of claim 21, wherein flowing the liquid polymeric fixative comprises extruding the liquid polymeric fixative through a cage.

23. The method of claim 21, wherein fusing the vertebrae comprises approaching the vertebrae using a transverse approach.

24. The method of claim 21, wherein fusing the vertebrae comprises approaching the vertebrae using a transverse approach.

25. The method of claim 21, wherein flowing the liquid polymeric fixative comprises preferentially directing the flow of the liquid polymeric fixative toward faces of the vertebrae.

26. A spinal fusion comprising a solid, load bearing polymeric fixative fixed to opposing faces of spinal vertebrae without hardware.

27. The spinal fusion of claim 26, wherein the solid, load bearing polymeric fixative comprises a polyurethane.

28. The spinal fusion of claim 26, further comprising a cage disposed between the opposing faces of the spinal vertebrae.

29. The spinal fusion of claim 26, wherein the solid, load bearing polymeric fixative comprises a disc-shaped solid member that is dimensioned by the opposing faces of the spinal vertebrae.
